(19) United States
(12) Patent Application Publication
(10) Pub. No.: US 2025/0033032 A1
(43) Pub. Date: Jan. 30, 2025

(54) SORBENT FOR DIALYSIS AND SORBENT SYSTEM FOR REGENERATIVE DIALYSIS

(71) Applicant: AWAK TECHNOLOGIES PTE LTD, Singapore (SG)

(72) Inventors: Suresha BELURE VENKATARAYA, Singapore (SG); Mandar Manohar GORI, Singapore (SG); Sanjay Kumar SINGH, Singapore (SG); Joel Preetham FERNANDES, Singapore (SG); Daniel Wei Teik TAN, Singapore (SG); Marcin Bartlomiej PAWLAK, Singapore (SG); Sridhar CHIRUMARRY, Singapore (SG); Vinod Kumar GADI, Singapore (SG); Jason Tze Chern LIM, Singapore (SG); Yue (Victoria) WANG, Singapore (SG); Peter HAYWOOD, Singapore (SG)

(21) Appl. No.: 18/713,886

(22) PCT Filed: Nov. 29, 2022
(86) PCT No.: PCT/SG2022/050867

§ 371 Date: May 28, 2024

(30) Foreign Application Priority Data
Nov. 30, 2021 (SG) 10202113299U

(51) Int. Cl. B01J 47/04; A61M 1/16; B01J 20/02; B01J 20/04; B01J 20/20; B01J 39/12; B01J 41/10

(52) U.S. Cl. CPC B01J 47/04; A61M 1/1654; B01J 20/0211; B01J 20/0292; B01J 20/041; B01J 20/043; B01J 20/20; B01J 39/12; B01J 41/10; B01J 2220/42; B01J 2220/62

(57) ABSTRACT

Disclosed herein is a material for use in sorbent-based dialysis, the material comprising: acidic and/or neutral cation exchange particles; alkaline anion exchange particles; and one or more of an alkali metal carbonate, a water insoluble alkaline earth metal carbonate, and a water insoluble polymeric ammonium carbonate. Also disclosed herein are uses of said material and its preparation.

### FIELD OF INVENTION

The present invention relates to a sorbent for dialysis as well as to a sorbent system for regenerative dialysis which may be, but is not limited to, haemodialysis, peritoneal dialysis, liver dialysis, lung dialysis, water purification and regeneration of biological fluids.

### BACKGROUND

The listing or discussion of a prior-published document in this specification should not necessarily be taken as an acknowledgement that the document is part of the state of the art or is common general knowledge.

Chronic kidney disease (CKD) often leads to an imbalance in serum bicarbonate and sodium concentration. Patients commonly suffer from low bicarbonate and low serum pH in the form of metabolic acidosis, while untreated CKD can lead to dangerously high serum sodium due to accumulation of dietary sodium intake. These imbalances present a severe risk towards the central nervous system and cardiovascular health. Therefore, a fundamental goal of dialysis is the correction of the serum sodium balance and the acid-base balance in order to maintain blood homeostasis.

In conventional peritoneal dialysis such as CAPD or APD, sodium is corrected by maintaining a negative concentration gradient between the dialysate (Na 132 mmol/L) and the patient's serum sodium concentration (approx. Na 138 mmol/L), whereby sodium is removed through diffusion from the blood to the dialysate. This concentration gradient is further heightened by transport of ultrafiltrate to the peritoneum, which is low in sodium and dilutes the dialysate further. Bicarbonate is corrected by maintaining a positive alkali balance (net transfer of alkali from dialysate to patient serum) using a high concentration of lactate ions (Lac 40 mmol/L) in the dialysate, which diffuse into the patient's bloodstream and are metabolized by the liver to bicarbonate. Therefore, in conventional peritoneal dialysis, sodium and bicarbonate are managed by somewhat different mechanisms and these mechanisms do not directly affect one another.

In contemporary sorbent dialysis systems consisting of Urease, zirconium phosphate (ZP) and hydrous zirconium oxide (HZO), the control of bicarbonate and sodium are directly related, presenting limitations in terms of concurrent optimization of Na⁺ and HCO₃₋ balance. The primary method of sodium control in sorbent dialysis is removal through ion exchange with hydrogen loaded ZP (ZP—H):

ZP—H+Na⁺→ZP—Na+H⁺

However, this ion exchange process only readily occurs in the presence of base, for example the bicarbonate ion:

ZP—H+HCO₃₋+Na⁺→ZP—Na+H₂O+CO₂

Depending on the overall pH of the dialysate, the CO₂ may be lost to the atmosphere and result in a net loss of alkali in the dialysate fluid. While exclusive use of an acidic H-loaded ZP may be suitable for control of sodium and removal of other unwanted cations such as ammonium, the subsequent loss of bicarbonate and low resultant pH would lead to a worse bicarbonate balance overall. This is illustrated in the solution mole fraction of aqueous carbonic acid, bicarbonate and carbonate vs pH depicted in FIG. 1 and in the solution mole fraction of aqueous ammonium and ammonia vs pH depicted in FIG. 2.

The effect of low pH and low bicarbonate is typically counterbalanced through addition of a basic salt to the sorbent, such as sodium bicarbonate, and/or use of an alkaline anion exchanger, for example OH-loaded HZO.

The limitation with the sodium bicarbonate approach is that this salt is readily soluble in the aqueous dialysis fluid, and therefore leads to a sharp increase in dialysate sodium and pH at the start of the therapy. The direct addition of a soluble sodium salt is counterproductive towards sodium control in this case. This is because in peritoneal dialysis a blood-dialysate concentration gradient is desirable in order to remove sodium from the patient. Furthermore, this approach does not offer a sustained increase in pH over the course of the therapy, which would be desirable from both the patient bicarbonate standpoint, as the stability of bicarbonate is pH dependent as mentioned earlier.

The use of alkaline HZO offer some advantages in that it helps to neutralize acidic dialysate and remove phosphate leached by ZP—H:

HZO—OH+H⁺+X⁻→HZO—X+H₂O(pH<<7,X=Cl,PO₄,F)

However, the quantity of HZO required to act as a buffer is not insignificant, and can affect the size and weight of the sorbent cartridge considerably. Moreover, the rate of reaction between HZO and H is rapid and so this buffer capacity is readily depleted, meaning that the pH and bicarbonate concentration are only maintained during the start of a therapy.

The current method of sodium control means that Na⁺ removal co-occurs with increased HCO₃₋ removal. Too much HCO₃₋ removal can cause metabolic acidosis, which can trigger many unhealthy symptoms and harm to the patients. There are alternative methods of addressing metabolic acidosis in current practice which could be used as an adjunct therapy, such as orally ingested sodium bicarbonate tablets, however this solution is confounded by the same problem; Na⁺ is added back to the blood system. Thus, there is a need for an improved method of bicarbonate management, specifically for sorbent dialysis, to serve as suitable alternative to sodium bicarbonate and alkaline HZO.

### SUMMARY OF INVENTION

Disclosed herein is a sorbent composition consisting of different percentages of neutral ZP (NZP), acidic ZP (AZP), alkaline HZO (NaHZO), as well as the substantially insoluble salts CaCO₃ and Ca(OH)₂. This surprisingly solves some or all of the problems identified above.

Aspects and embodiments of the invention are provided in the following numbered clauses.
1. A material for use in sorbent-based dialysis, the material comprising:
   acidic and/or neutral cation exchange particles;
   alkaline anion exchange particles; and
   one or more of an alkali metal carbonate, a water insoluble alkaline earth metal carbonate, and a water insoluble polymeric ammonium carbonate.
2. The material according to Clause 1, wherein the material further comprises one or both of Ca(OH)₂, and Mg(OH)₂.
3. The material according to Clause 1 or Clause 2, wherein the acidic and/or neutral cation exchange particles are acidic and/or a neutral water-insoluble metal phosphate, optionally wherein the metal is selected from one or more of the group consisting of titanium, zirconium, and hafnium.
4. The material according to Clause 3, wherein the metal is zirconium.
5. The material according to any one of the preceding clauses, wherein the alkaline anion exchange particles comprise an amorphous and partly hydrated, water-insoluble metal oxide in its: hydroxide-; and/or carbonate-; and/or acetate-; and/or lactate- counter-ion form, wherein the metal is selected from one or more of the group consisting of titanium, zirconium, and hafnium, optionally wherein the anion exchange particles are alkaline hydrous zirconium oxide.
6. The material according to any one of the preceding clauses, wherein:
   (a) the water insoluble alkaline earth metal carbonate is selected from one or more of the group consisting of CaCO₃ and MgCO₃₋; and/or
   (b) the alkali metal carbonate is K₂CO₃; and/or
   (c) the water insoluble polymeric ammonium carbonate is selected from one or more of the group consisting of sevelamer carbonate, polymer-bound tetra-alkyl ammonium carbonate, and 3-(trialkyl ammonium) alkyl (e.g. propyl) functionalised silica gel carbonate.
7. The material according to any one of the preceding clauses, wherein the material comprises:
   from 30 to 79 wt % of acidic and/or neutral cation exchange particles;
   from 20 to 65 wt % of alkaline anion exchange particles;
   one or more of an alkali metal carbonate, a water insoluble alkaline earth metal carbonate, and a water insoluble polymeric ammonium carbonate in a total amount from 0.1 to 10 wt %; and
   one or both of Ca(OH)₂, and Mg(OH)₂ in a total amount of from 0 to 5 wt %.
8. The material according to Clause 7, wherein the material comprises:
   from 31 to 75 wt % of acidic and/or neutral cation exchange particles;
   from 23 to 63 wt % of alkaline anion exchange particles;
   one or more of an alkali metal carbonate, a water insoluble alkaline earth metal carbonate, and a water insoluble polymeric ammonium carbonate in a total amount of from 0.1 to 5 wt %; and
   one or both of Ca(OH)₂, and Mg(OH)₂ in a total amount of from 0 to 4 wt %.
9. The material according to Clause 7 or Clause 8, wherein the material comprises:
   from 50 to 64 wt % of acidic and/or neutral cation exchange particles;
   from 35 to 45 wt % of alkaline anion exchange particles; and
   one or more of an alkali metal carbonate, a water insoluble alkaline earth metal carbonate, and a water insoluble polymeric ammonium carbonate a total amount of from 0.3 to 5 wt %.
10. The material according to Clause 9, wherein the material comprises:
   from 53 to 60 wt % of acidic and/or neutral cation exchange particles;
   from 39 to 44 wt % of alkaline anion exchange particles; and
   one or more of an alkali metal carbonate, a water insoluble alkaline earth metal carbonate, and a water insoluble polymeric ammonium carbonate a total amount of from 0.5 to 3 wt %.
11. The material according to Clause 7 or Clause 8, wherein the material comprises:
   from 45 to 59 wt % of acidic and/or neutral cation exchange particles;
   from 40 to 54 wt % of alkaline anion exchange particles; and
   one or more of an alkali metal carbonate, a water insoluble alkaline earth metal carbonate, and a water insoluble polymeric ammonium carbonate a total amount of from 0.5 to 5 wt %.
12. The material according to Clause 11, wherein the material comprises:
   from 48 to 56 wt % of acidic and/or neutral cation exchange particles;
   from 42 to 50 wt % of alkaline anion exchange particles; and
   one or more of an alkali metal carbonate, a water insoluble alkaline earth metal carbonate, and a water insoluble polymeric ammonium carbonate a total amount of from 1 to 2 wt %.
13. The material according to Clause 7 or Clause 8, wherein the material comprises:
   from 50 to 70 wt % of acidic and/or neutral cation exchange particles;
   from 30 to 49 wt % of alkaline anion exchange particles;
   from 0.2 to 3 wt % one or more of an alkali metal carbonate, a water insoluble alkaline earth metal carbonate, and a water insoluble polymeric ammonium carbonate; and
   one or both of Ca(OH)₂, and Mg(OH)₂ in a total amount of from 0.2 to 2 wt %.
14. The material according to Clause 13, wherein the material comprises:
   from 53 to 67 wt % of acidic and/or neutral cation exchange particles;
   from 33 to 46 wt % of alkaline anion exchange particles;
   from 0.2 to 2 wt % one or more of an alkali metal carbonate, a water insoluble alkaline earth metal carbonate, and a water insoluble polymeric ammonium carbonate a total amount of; and
   one or both of Ca(OH)₂, and Mg(OH)₂ in a total amount of from 0.2 to 1.5 wt %.
15. The material according to any one of the preceding clauses, wherein the material is one in which:
   the cation exchange particles are an acidic and/or a neutral water-insoluble metal phosphate;
   anion exchange particles are an alkaline hydrous zirconium oxide; and
   the one or more of an alkali metal carbonate, a water insoluble alkaline earth metal carbonate, and a water insoluble polymeric ammonium carbonateis CaCO₃ and/or MgCO₃, optionally wherein the material further comprises Ca(OH)₂.
16. The material according to any one of the preceding clauses, wherein the material further comprises an organic compounds absorber, wherein the organic compounds absorber is present in an amount of from 10 to 40 wt % relative to the total weight of the components listed in Clause 1, optionally wherein the organic compounds absorber is present in an amount of from 15 to 25 wt %, such as from 18 to 23 wt %, such as from 19 to 21 wt % relative to the total weight of the components listed in Clause 1.
17. The material according to Clause 16, wherein the organic compounds absorber is activated carbon.
18. The material according to any one of the preceding clauses, wherein the material further comprises neutral hydrous zirconium oxide, wherein the neutral hydrous zirconium oxide is present in an amount of from 0.1 to 10 wt % relative to the total weight of the components listed in Clause 1, optionally wherein the neutral hydrous zirconium oxide is present in an amount of from 0.5 to 5 wt % relative to the total weight of the components listed in Clause 1.
19. The material according to any one of Clauses 4 and 5 to 18, as dependent upon Clause 4, wherein both an acidic zirconium phosphate and a neutral zirconium phosphate are present and the acidic zirconium phosphate is present in an amount of from 55 to 80 wt % of the total amount of zirconium phosphate in the material, with the neutral zirconium phosphate supplying the balance to 100 wt %.
20. The material according to Clause 19, wherein:
   (a) the acidic zirconium phosphate is present in an amount of from 59 to 70 wt % of the total amount of zirconium phosphate in the material, with the neutral zirconium phosphate supplying the balance to 100 wt %; or
   (b) the acidic zirconium phosphate is present in an amount of from 75 to 78 wt % of the total amount of zirconium phosphate in the material, with the neutral zirconium phosphate supplying the balance to 100 wt %.
21. The material according to any one of the preceding clauses, wherein:
   (a) all of the components are intermixed together to provide a single layer of material; or
   (b) the one or more of an alkali metal carbonate, a water insoluble alkaline earth metal carbonate, and a water insoluble polymeric ammonium carbonate, and, when present, the metal hydroxide are intermixed with the cation exchange particles to form a first layer, with the anion exchange particles provided as a second layer.
22. The material according to any one of the preceding clauses, which comprises one or both of a water insoluble alkaline earth metal carbonate, and a water insoluble polymeric ammonium carbonate.
23. A cartridge for use in sorbent dialysis, the cartridge comprising a material as described in any one of Clauses 1 to 22.

### DRAWINGS

FIG. 1: Solution mole fraction of aqueous carbonic acid, bicarbonate and carbonate vs pH.

FIG. 2: Solution mole fraction of aqueous ammonium and ammonia vs pH.

FIG. 3: Schematic of Sorbent cartridge according to an embodiment of the invention and used in the examples disclosed herein.

FIG. 4: Experimental setup.

FIG. 5: Different composition amounts of Ca(OH)₂ and its overall contribution to the dialysate pH profile during 7-hour treatment

FIG. 6: Depicts a sorbent cartridge according to embodiments of the invention.

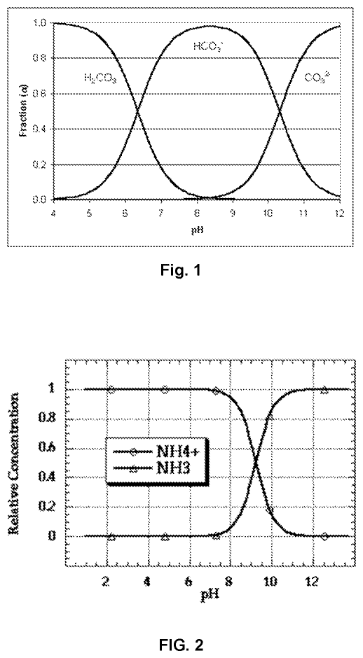

### DESCRIPTION

It has been surprisingly found that it is possible to modify the bicarbonate and sodium concentrations in a dialysate undergoing sorbent-based dialysis through addition of specific metal carbonate salts and/or specific metal hydroxide salts.

Thus, in a first aspect of the invention, there is provided a material for use in sorbent-based dialysis, the material comprising:
acidic and/or neutral cation exchange particles;
alkaline anion exchange particles; and
one or more of an alkali metal carbonate, a water insoluble alkaline earth metal carbonate, and a water insoluble polymeric ammonium carbonate.

In certain embodiments, the material above may further comprise one or both of Ca(OH)₂, and Mg(OH)₂.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

The term “sorbent” as used herein broadly refers to a class of materials characterized by their ability to absorb the desired matter of interest.

The term “metabolic wastes” in the context of this specification, means any constituents, typically toxic constituents, within a dialysate that are produced by metabolism and which are desirable to be removed in a dialysate detoxification process. Typical metabolic wastes include, but are not limited to phosphates, urea, creatinine and uric acid.

The term “essential cations” as used herein refers to cations other than sodium ions that are present in dialysis solutions and are essential for their safe and effective use. These ions are generally calcium and magnesium ions but potassium ions may also be present. Calcium, magnesium and potassium are removed by the sorbent and need to be reintroduced to regenerated dialysate to reconstitute the dialysate.

The term “cation equivalents” or “total cation equivalents” refers to the sum of all positive charge equivalents, except protons in a solution. It is measured in mEq/L.

The term “sodium” or the symbol “Na” may be used in the specification to refer to sodium ions rather than to the element itself, as would be well understood by the person skilled in the art. Accordingly, the terms “sodium”, “Na”, “sodium ions” and “Na*” are used interchangeably. Likewise, the terms “calcium”, “magnesium” and “potassium” or the symbols “Ca”, “Mg” and “K” may be used in the specification to refer to calcium ions, magnesium ions and potassium ions, respectively.

The term a “source of spent dialysate” as used herein is a reference to a source of dialysate however it is produced. The source may be any source of spent fluid where the regeneration of biological fluids takes place by exchange across a membrane. If, for example, the dialysis process is haemodialysis then the source of the spent dialysate will be a dialyser in a haemodialysis apparatus. In such apparatus streams of blood from a patient and dialysate are in counter-current flow, and exchange takes place across a membrane separating the streams. Alternatively, it may be a patient as, for example, in peritoneal dialysis where dialysate is introduced to a patient's peritoneal cavity for exchange to take place.

The term “cation exchange particles” as used herein refers to particles capable of capturing or immobilizing cationic or positively charged species when contacted with such species, typically by passing a solution of the positively charged species over the surface of the particles.

The term “anion exchange particles” as used herein refers to particles capable of capturing or immobilizing anionic or negatively charged species when contacted with such species, typically by passing a solution of the negatively charged species over the surface of the particles.

The term “uremic toxin-treating enzyme” as used herein refers to an enzyme able to react with a uremic toxin as a substrate. For example, the uremic toxic-treating enzyme may be an enzyme able to react with urea as a substrate, with uric acid as a substrate, or with creatinine as a substrate. Uremic enzymes can be determined to have this function in vitro, for example, by allowing the enzyme to react with a uremic toxin in solution and measuring a decrease in the concentration of the uremic toxin. Examples of uremic toxin-treating enzymes include, but are not limited to, ureases (which react with urea), uricases (which react with uric acid), or creatininases (which react with creatinine).

The term “uremic toxin” as used herein refers to one or more compounds comprising waste products, for example, from the breakdown of proteins, nucleic acids, or the like, as would be well understood by the person skilled in the art. Non-limiting examples of uremic toxins include urea, uric acid, creatinine, and beta-2 (β₂) microglobulin. In healthy individuals, uremic toxins are usually excreted from the body through the urine. However, in certain individuals, uremic toxins are not removed from the body at a sufficiently fast rate, leading to uremic toxicity, i.e. a disease or condition characterized by elevated levels of at least one uremic toxin with respect to physiologically normal levels of the uremic toxin. Non-limiting examples of disorders associated with uremic toxins include renal disease or dysfunction, gout, and uremic toxicity in subjects receiving chemotherapy.

The term “uremic toxin-treating enzyme particles” as used herein refers to a uremic toxin-treating enzyme in particle form. The enzymes may be immobilized by way of a covalent or physical bond to a biocompatible solid support, or by cross-linking, or encapsulation, or any other means.

The term “soluble source” as used herein refers to a compound distinct from other components of the sorbent which may be added to and mixed with the other components, or be present as a separate layer or in a compartment separate from other sorbent components. It will usually be added to the sorbent in the form of solid particles which intermix with other solid particles in the sorbent.

The term “biocompatible” as used herein refers to the property of a material that does not cause adverse biological reactions to the human or animal body.

The term “homogeneous” as used herein refers to a substantially homogeneous mixture, meaning a mixture have the same proportions of the various components throughout a given sample, creating a consistent mixture. The composition of the mixture is substantially the same overall, although it will be appreciated that in mixing solid particles there may be regions in a sample where mixing is not complete.

The term “particle size” refers to the diameter or equivalent diameter of the particle. The term “average particle size” means that a major amount of the particles will be close to the specified particle size although there will be some particles above and some particles below the specified size. The peak in the distribution of particles will have a specified size. Thus, for example, if the average particle size is 50 microns, some particles which are larger and some particles which are smaller than 50 microns will exist.

The terms “regenerate” or “regenerated” as used herein refer to the action of detoxifying dialysate by destruction and/or absorption of uremic toxins by a sorbent.

The term “regenerated dialysate” as used herein refers to dialysate which has been detoxified by destruction and/or absorption of uremic toxins by a sorbent.

The term “reconstitute” or “reconstituted” as used herein refer to the action of converting regenerated dialysate to essentially the same state and chemical composition as fresh dialysate prior to dialysis.

The term “reconstituted dialysate” as used herein refers dialysate which has been converted to essentially the same state and chemical composition as fresh dialysate prior to dialysis.

The term “predominantly” as used herein is intended to represent a situation or state which occurs for the most part or principally, while not excluding the possibility that some amount of another situation or state also occurs to a minimal extent. For example, it may be >80% or >90% or >95% or greater than 99%. For the avoidance of doubt, the possibility that only that situation or state occurs, to the exclusion of all others, is covered by the term.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, typically means±5% of the stated value, more typically +/−4% of the stated value, more typically±3% of the stated value, more typically, +/−2% of the stated value, even more typically±1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The acidic and/or neutral water-insoluble metal phosphate may be any metal phosphate which has a solubility not higher than 10 mg/L in water. Examples of suitable acidic and/or neutral water-insoluble metal phosphates include those where the metal is selected from the group consisting of titanium, zirconium, hafnium and combinations thereof. In particular embodiments that may be mentioned herein, the acidic and/or neutral water-insoluble metal phosphate may be acidic and/or neutral zirconium phosphate. The process for the preparation of neutral zirconium phosphate and acidic zirconium phosphate is similar, except that the buffer pH and its ratio with respect to sodium zirconium carbonate is changed to match the desired pH value. Both are prepared by mixing sodium zirconium carbonate with a phosphate buffer having the desired pH value and in an appropriate ratio, which can readily be determined by a skilled person.

When used herein, the term “and/or” when applied to two specific materials, such as “acidic and/or neutral zirconium phosphate” is intended to allow combinations of the mentioned components or for the individual use of said component. That is, the term “acidic and/or neutral zirconium phosphate” covers embodiments where:
only acidic zirconium phosphate is present;
only neutral zirconium phosphate is present; or
both acidic and neutral zirconium phosphate are present.

Acidic and/or neutral water-insoluble metal phosphates may be used as ion-exchange materials and are particularly useful as a sorbent material in regenerative kidney dialysis. For example, zirconium phosphate in the sodium or hydrogen form serves as a cation exchanger and absorbs cations such as ammonium (NH₄⁺), calcium (Ca²⁺), potassium (K⁺), and magnesium (Mg²⁺). In exchange for absorbing these cations, zirconium phosphate releases two other cations, sodium (Na⁺) and hydrogen (H⁺). Neutral zirconium phosphate helps to maintain an appropriate in-situ pH when it is mixed with acidic zirconium phosphate. Without wishing to be bound by theory, it is believed that neutral zirconium phosphate helps to maintain the bicarbonate balance of the dialysate along with CaCO₃ and Ca(OH)₂.

In an embodiment the acidic and/or neutral water-insoluble metal phosphate are configured to exchange ammonium ions for predominantly hydrogen ions and to exchange essential cations for sodium ions by setting them to low pH during synthesis. To optimise this property, the cation exchange particles are typically set to low pH and low sodium loading during synthesis.

In an embodiment the cation exchanger is synthesised in the presence of an acid. The pH is set by adjustment to a desired level, such as by titration with a base such as sodium hydroxide to raise the pH to a level which provides the desired differential exchange behaviour. The titration also serves to provide the cation exchange particles with a sufficient loading of sodium to enable the desired exchange of sodium for calcium, magnesium and potassium. In an embodiment the cation exchange material is zirconium phosphate. This may be synthesised in conventional processes such, for example, from Basic Zirconium Sulphate (BZS) or from zirconium carbonate by reaction with phosphoric acid. If other acids are used a source of the phosphate group must be provided. Typically, the pH is set to be in the range of 3.5 to 5.0, advantageously about 4.5, by titration of the reaction product with a base.

Acidic zirconium phosphate may also be prepared, for example, by following the methods disclosed in U.S. Pat. No. 6,818,196, which is incorporated in its entirety by reference herein. Briefly, acidic zirconium phosphate can be prepared by heating zirconium oxychloride (ZOC) with soda ash to form sodium zirconium carbonate, and treating the sodium zirconium carbonate with caustic soda to form alkaline hydrous zirconium oxide. An aqueous slurry of the alkaline hydrous zirconium oxide can then be heated, while adding phosphoric acid. An aqueous slurry of the acidic zirconium phosphate can also be titrated with a basic agent, such as caustic soda, until a desired pH is reached, for example, a pH of from about 5 to about 7.

The acidic and/or neutral zirconium phosphate particles may have an average particle size in the range of from about 10 microns to about 1000 microns, about 100 microns to about 900 microns, about 200 microns to about 900 microns, about 300 microns to about 800 microns, about 400 microns to about 700, 500 microns to about 600 microns, about 25 microns to about 200 microns or from about 25 microns to about 150 microns or from about 25 microns to about 80 microns or from about 25 microns to about 50 microns or from about 50 microns to about 100 microns or from about 125 microns to about 200 microns, or from about 150 microns to about 200 microns, or from about 100 microns to about 175 microns, or from about 100 microns to about 150 microns or from about 150 microns to about 500 microns, or from about 250 microns to about 1000 microns. The acidic and/or neutral zirconium phosphate particles may be immobilized on any known support material, which can provide immobilization for the zirconium phosphate particles. In one embodiment, the support material may be a biocompatible substrate. In one embodiment, the immobilization of the acidic and/or neutral zirconium phosphate particles is a physical compaction of the particles into a predetermined volume. In one embodiment, the immobilization of the acidic and/or neutral zirconium phosphate particles is achieved by sintering zirconium phosphate, or a mixture of zirconium phosphate and a suitable ceramic material. The biocompatible substrate may be a homogeneous substrate made up of one material or a composite substrate made up of at least two materials

The anion exchange particles may comprise of an amorphous and partly hydrated, water-insoluble metal oxide in its hydroxide-, carbonate-, acetate-, and/or lactate- counter-ion form, wherein the metal may be selected from the group consisting of titanium, zirconium, hafnium and combinations thereof. In one embodiment, the metal is zirconium. The anion exchange particles may be zirconium oxide particles. Preferably, the anion exchange particles are hydrous zirconium oxide particles.

Alkaline hydrous zirconium oxide, or NaHZO, means the alkaline form of hydrous zirconium oxide (ZrO(OH)₂), in which the zirconium oxide is hydroxylated. NaHZO may have the following chemical and physical properties:
Composition: Na⁺_{z},ZrO₂ (OH⁻)_{y}·ₙH₂O
Ion-exchange formula: ZrO₂·OH³¹
wherein x for Na⁺ is 1, y for OH— may be from 2 to 4 and n for H₂O may be from 4 to 6, and x, y, and n may be any decimal in these ranges and can optionally be above or below these ranges. The NaHZO can have a Na⁺ content Na:Zr₂ (molar ratio) in a range of, for example, from about 0.5:1.5 to about 1.5:0.5, such as about 1:1, and/or have a hydroxyl ion content in a range of, for example, from about 3 to about 12 mEq OH⁻/10 g NaHZO, from about 5 to about 10 mEq OH⁻/10 g NaHZO, or from about 6 to about 9 mEq OH⁻/10 g NaHZO. The NaHZO may have a pH in water (1 g/100 mL) of, for example, from about 7 to about 14, from about 9 to about 12, or from about 10 to about 11. As noted in the formulae above, the purpose of the alkaline hydrous zirconium oxide is to release hydroxide ions.

The alkaline hydrous zirconium oxide particles may have an average particle size in the range of from about 10 microns to about 1000 microns, about 100 microns to about 900 microns, about 200 microns to about 900 microns, about 300 microns to about 800 microns, about 400 microns to about 700, 500 microns to about 600 microns, about 10 microns to about 200 microns or from about 10 microns to about 100 microns or from about 10 microns to about 30 microns or from about 10 microns to about 20 microns or from about 20 microns to about 50 microns or from about 25 microns to about 50 microns or from about 30 microns to about 50 microns or from about 40 microns to about 150 microns or from about 80 microns to about 120 microns or from about 160 microns to about 180 or from about 25 microns to about 250 or from about 250 microns to about 500 or from about 250 microns to about 1000. The zirconium oxide particles may be immobilized on any known support material which can provide immobilization for the zirconium oxide particles. In one embodiment, the immobilization of the zirconium oxide particles may be a physical compaction of the particles into a predetermined volume. In one embodiment, the immobilization of the zirconium oxide particles is achieved by sintering zirconium oxide, or a mixture of zirconium oxide and a suitable ceramic material. In one embodiment, the support material is a biocompatible substrate. The biocompatible material may be a carbohydrate-based polymer, an organic polymer, a polyamide, a polyester, a polyacrylate, a polyether, a polyolefin or an inorganic polymeric or ceramic material. The biocompatible substrate may be at least one of cellulose, Eupergit, silicon dioxide, nylon, polycaprolactone and chitosan.

In one embodiment, the alkaline hydrous zirconium oxide particles may be replaced by any particles that are able to absorb phosphate ions and other anions. Preferably, the particles are able to absorb anions selected from the group comprising ions of phosphate, fluoride, nitrate and sulphate. The zirconium oxide particles may also release ions such as acetate, lactate, bicarbonate and hydroxide in exchange for the anions absorbed.

Alkaline hydrous zirconium oxide can be prepared by the reaction of a zirconium salt, for example, BZS, or its solution in water with an alkali metal (or alkali metal compound) at ambient temperature, to form an alkaline hydrous zirconium oxide precipitate. The alkaline hydrous zirconium oxide particles can be filtered and washed until the anions of the zirconium salt are completely removed, and then air dried, or dried in an oven at mild temperature to a moisture level, for instance, of from about 30 to 40 weight percent LOD or lower, to form a free-flowing powder. Other LODs can be achieved, although higher temperature and/or long drying time (e.g. 24-48 hrs) to achieve a lower moisture level (i.e., <20 weight percent LOD) can convert the zirconium-hydroxide bond to a zirconium-oxide bond and reduce the adsorption capacity as well as alkalinity of the anion-exchange material.

Alkaline hydrous zirconium oxide can also be prepared, for example, by following the methods disclosed in U.S. Patent Application Publication 2006/0140844, which is incorporated in its entirety by reference herein, in combination with the teachings provided herein. Briefly, this method of preparing alkaline hydrous zirconium oxide involves adding an aqueous solution of ZOC, titrated with concentrated HCl, to an aqueous solution of caustic soda. The HCl addition can prevent excessive gelation during the precipitation process as well as to promote particle growth. Neutral hydrous zirconium oxide can be prepared by modifying the procedure described herein for the manufacture of basic zirconium oxide. For example, this may be achieved by controlling the pH of the aqueous slurry formed by treatment of sodium zirconium carbonate and sodium hydroxide, so as to arrive at a neutral hydrous zirconium oxide.

As noted above, an essential component of the sorbent disclosed herein is the presence of: a water insoluble alkaline earth metal carbonate, an alkali metal carbonate, a water insoluble polymeric ammonium carbonate, and combinations thereof. In particular embodiments that may be mentioned herein:
(a) the water insoluble alkaline earth metal carbonate may be selected from one or more of the group consisting of CaCO₃ and MgCO₃;
(b) the alkali metal carbonate may be K₂CO₃; and/or
(c) the water insoluble polymeric ammonium carbonate may be selected from one or more of the group consisting of sevelamer carbonate, polymer-bound tetra-alkyl ammonium carbonate, and 3-(trialkyl ammonium) alkyl (e.g. propyl) functionalised silica gel carbonate.

When used herein, the term alkyl may refer to a linear or branched C₁ to C₆ alkyl group and may include methyl, ethyl, propyl, isopropyl, n-butyl, i-butyl and t-butyl groups, amongst others.

Without wishing to be bound by theory, it is believed that the: water insoluble alkaline earth metal carbonate; alkali metal carbonate; a water insoluble polymeric ammonium carbonate; and combinations thereof in the sorbent act as a direct source of bicarbonate and functions as a mild pH buffer. Similarly, when Ca(OH)₂ and Ca(OH)₂ are included in the formulation, they are believed to act in a similar manner. For example, when CaCO₃ (or MgCO₃) is present, it acts as a direct source of bicarbonate and functions as a mild pH buffer, while Ca(OH)₂ (or Mg(OH)₂; when present) is more basic and helps to increase dialysate pH further. A high pH facilitates the conversion of CO₂, generated during urea hydrolysis or by reaction with ZP, to bicarbonate.

The corresponding overall chemical reactions can be presented as shown below for the calcium species mentioned above,

CaCO₃(s)+H₂O(I)+CO₂(g)<=>Ca²⁺(aq)+2HCO₃₋(aq)

Ca(OH)₂(s)+H₂O(I)+2CO₂(g)<=>Ca²⁺(aq)+2HCO₃₋(aq)

As will be appreciated, similar reactions occur when the other materials mentioned above are used instead of these calcium species. Conversion of bicarbonate depends on equilibrium pH, and dissociation constant and dissolution rate of CaCO₃ and Ca(OH)₂.

In a low urea cartridge configuration, CaCO₃ (or MgCO₃) plays a more significant role in modulating the HCO₃₋ balance, given the fact that in the case of a low serum urea patient, less CO₂ is produced by urea hydrolysis. Hence, there is less CO₂ to be converted to HCO₃—, and therefore less capacity to ameliorate acidosis in the patient. In such a scenario, additional CaCO₃ (or MgCO₃) serves as direct source of HCO₃₋, while helping to modulate the pH and maintain the stability of HCO₃ or CO₂ already present in solution. When used herein, the term “low urea cartridge configuration” refers to a cartridge that is designed to clear a urea concentration of from 3 mM to 5.5 mM.

In a high urea cartridge configuration, Ca(OH)₂ plays a more significant role in modulating the HCO₃₋ balance. In the case where a patient with high serum urea is treated, more CO₂ will be present in the dialysate due to an increased quantity of urea being hydrolysed.

Urea+H₂O→urease→2NH₃+CO₂

When used herein, the term “high urea cartridge configuration” refers to a cartridge that is designed to clear a urea concentration of from 5 mM to 8 mM.

The effect of Ca(OH)₂ on the bicarbonate balance is perhaps best shown in Examples 6 and 7, where the addition of 2.5 g of Ca(OH)₂ to the sorbent composition results in a higher bicarbonate balance than obtained for the composition of Example 6, where no Ca(OH)₂ is present.

The addition of Ca(OH)₂ helps to improve the pH level for the dialysate solution, which facilitates the conversion of CO₂ to HCO₃₋.

Although Ca(OH)₂ and CaCO₃ are conducive for overall HCO₃₋ balance, adding too much might lead to less Na⁺ and ammonium removal. When Ca(OH)₂ and CaCO₃ dissolve, Ca₂⁺ will be released into the dialysate. Ca₂⁺ will then be preferentially bound by zirconium phosphate (or other water insoluble metal phosphate), taking up some ion exchange capacity which would have been used for sodium and ammonia control. As such, when designing an optimal sorbent composition including Ca(OH)₂ and CaCO₃, factors such as pH, impact on Na⁺ balance, HCOs balance and ammonium binding capacity will all need to be considered.

In some embodiments of the invention that may be mentioned herein, the carbonate salt present in the sorbent may be an insoluble carbonate salt. In other words, in some embodiments of the invention that may be mentioned herein, the material may comprise one or more of a water insoluble alkaline earth metal carbonate, and a water insoluble polymeric ammonium carbonate. This may advantageously prevent rapid dissolution of the carbonate salt during dialysis, ensuring that the sorbent provides a steady source of bicarbonate throughout the entire duration of a sorbent treatment. In consequence, the use of a water insoluble carbonate is believed to mean that the sorbent is able to provide a steady supply of bicarbonate ions throughout the duration of a dialysis treatment without causing a sharp increase in sodium concentration or pH at the start of the treatment.

Particle size can influence dissolution rate, and hence can be a factor to control the conversion rate of bicarbonate, sorbent pH, and dialysate pH. This is a design factor to be considered. Any suitable particle size for CaCO₃ may be used herein. For example, from about 1 μm to about 100 μm. A suitable particle size distribution for CaCO₃ particles may be on in which the D90 may be about 38 μmm the D50 may be about 16 μm, and the D10 may be about 5 μm. Any suitable particle size for Ca(OH)₂ may be used herein. For example, from about 1 μm to about 80 μm. A suitable particle size distribution for Ca(OH)₂ particles may be on in which the D90 may be about 30 μmm the D50 may be about 11 μm, and the D10 may be about 3 μm.

Any suitable amount of the components mentioned above may be used in the sorbents disclosed herein. For example, the material may be one in which the material comprises:
from 30 to 79 wt % of acidic and/or neutral cation exchange particles;
from 20 to 65 wt % of alkaline anion exchange particles;
one or more of an alkali metal carbonate, a water insoluble alkaline earth metal carbonate, and a water insoluble polymeric ammonium carbonate in a total amount from 0.1 to 10 wt %; and
one or both of Ca(OH)₂, and Mg(OH)₂ in a total amount of from 0 to 5 wt %. In more particular embodiments, this may be a material that comprises:
from 30 to 79 wt % of an acidic and/or a neutral zirconium phosphate;
from 20 to 65 wt % of an alkaline hydrous zirconium oxide;
from 0.1 to 10 wt % of CaCO₃ and/or MgCO₃₋; and
from 0 to 5 wt % of Ca(OH)₂.

For example, the material may be one in which the material comprises:
from 31 to 75 wt % of acidic and/or neutral cation exchange particles;
from 23 to 63 wt % of alkaline anion exchange particles;
one or more of an alkali metal carbonate, a water insoluble alkaline earth metal carbonate, and a water insoluble polymeric ammonium carbonate in a total amount of from 0.1 to 5 wt %; and
one or both of Ca(OH)₂, and Mg(OH)₂ in a total amount of from 0 to 4 wt %. In more particular embodiments, the sorbent may be one that comprises:
from 31 to 75 wt % of an acidic and/or a neutral zirconium phosphate;
from 23 to 63 wt % of an alkaline hydrous zirconium oxide;
from 0.1 to 5 wt % of CaCO₃ and/or MgCO₃; and
from 0 to 4 wt % of Ca(OH)₂.

The exact design of the material disclosed herein may be modified depending on the urea concentrations expected to be encountered in the dialysate of the subject that is to be treated. For example, in a subject that may be expected to have a low concentration (e.g. from 3 to 5.5 mM) of urea, then the material may be one in which the material comprises:
from 50 to 64 wt % of acidic and/or neutral cation exchange particles;
from 35 to 45 wt % of alkaline anion exchange particles; and
one or more of an alkali metal carbonate, a water insoluble alkaline earth metal carbonate, and a water insoluble polymeric ammonium carbonate a total amount of from 0.3 to 5 wt %. For example, the sorbent may be one that comprises:
from 50 to 64 wt % of an acidic or a neutral water-insoluble metal phosphate;
from 35 to 45 wt % of an alkaline hydrous zirconium oxide; and
from 0.3 to 5 wt % of CaCO₃ and/or MgCO₃.

For example, the material may be one in which the material comprises:
from 53 to 60 wt % of acidic and/or neutral cation exchange particles;
from 39 to 44 wt % of alkaline anion exchange particles; and
one or more of an alkali metal carbonate, a water insoluble alkaline earth metal carbonate, and a water insoluble polymeric ammonium carbonate a total amount of from 0.5 to 3 wt %. For example, the sorbent may be one that comprises:
from 53 to 60 wt % of an acidic or a neutral water-insoluble metal phosphate;
from 39 to 44 wt % of an alkaline hydrous zirconium oxide; and
from 0.5 to 3 wt % of CaCO₃ and/or MgCO₃.

Alternatively, a suitable material for use in a low urea concentration may be one in which the material comprises:
from 45 to 59 wt % of acidic and/or neutral cation exchange particles;
from 40 to 54 wt % of alkaline anion exchange particles; and
one or more of an alkali metal carbonate, a water insoluble alkaline earth metal carbonate, and a water insoluble polymeric ammonium carbonate a total amount of from 0.5 to 5 wt %. For example, the sorbent may be one that comprises:
from 45 to 59 wt % of an acidic and/or a neutral water-insoluble metal phosphate;
from 40 to 54 wt % of an alkaline hydrous zirconium oxide; and
from 0.5 to 5 wt % of CaCO₃ and/or MgCO₃.

For example, the material may be one in which the material comprises:
from 48 to 56 wt % of acidic and/or neutral cation exchange particles;
from 42 to 50 wt % of alkaline anion exchange particles; and
one or more of an alkali metal carbonate, a water insoluble alkaline earth metal carbonate, and a water insoluble polymeric ammonium carbonate a total amount of from 1 to 2 wt %. For example, the sorbent may be one that comprises:
from 48 to 56 wt % of an acidic and/or a neutral water-insoluble metal phosphate;
from 42 to 50 wt % of an alkaline hydrous zirconium oxide; and
from 1 to 2 wt % of CaCO₃ and/or MgCO₃.

In a subject that may be expected to have a high concentration (e.g. from 5 to 8 mM) of urea, then the material may be one in which the material comprises:
from 50 to 70 wt % of acidic and/or neutral cation exchange particles;
from 30 to 49 wt % of alkaline anion exchange particles;
from 0.2 to 3 wt % one or more of an alkali metal carbonate, a water insoluble alkaline earth metal carbonate, and a water insoluble polymeric ammonium carbonate a total amount of from 0.2 to 3 wt %; and
one or both of Ca(OH)₂, and Mg(OH)₂ in a total amount of from 0.2 to 2 wt %. For example, the sorbent may be one that comprises:
from 50 to 70 wt % of an acidic and/or a neutral water-insoluble metal phosphate;
from 30 to 49 wt % of an alkaline hydrous zirconium oxide;
from 0.2 to 3 wt % of CaCO₃ and/or MgCO₃; and
from 0.2 to 2 wt % of Ca(OH)₂.

For example, the material may be one in which the material comprises:
from 53 to 67 wt % of acidic and/or neutral cation exchange particles;
from 33 to 46 wt % of alkaline anion exchange particles;
one or more of an alkali metal carbonate, a water insoluble alkaline earth metal carbonate, and a water insoluble polymeric ammonium carbonate a total amount of from 0.2 to 2 wt %; and
one or both of Ca(OH)₂, and Mg(OH)₂ in a total amount of from 0.2 to 1.5 wt %. For example, the sorbent may be one that comprises:
from 53 to 67 wt % of an acidic and/or a neutral water-insoluble metal phosphate;
from 33 to 46 wt % of an alkaline hydrous zirconium oxide;
from 0.2 to 2 wt % of CaCO₃ and/or MgCO₃; and
from 0.2 to 1.5 wt % of Ca(OH)₂.

In particular embodiments of the above, the acidic and/or neutral water-insoluble metal phosphate may be and acidic and/or neutral zirconium phosphate.

In particular embodiments that may be mentioned herein:
the cation exchange particles are an acidic and/or a neutral water-insoluble metal phosphate an alkaline hydrous zirconium oxide;
anion exchange particles are; and
the one or more of an alkali metal carbonate, a water insoluble alkaline earth metal carbonate, and a water insoluble polymeric ammonium carbonate is CaCO₃ and/or MgCO₃, optionally wherein the material further comprises Ca(OH)₂.

The sorbent may be prepared in any suitable manner. For example, all of the components may be intermixed together to provide a single layer of material. Alternatively, the one or more of an alkali metal carbonate, a water insoluble alkaline earth metal carbonate, and a water insoluble polymeric ammonium carbonate, and, when present, the metal hydroxide may be intermixed with the cation exchange particles to form a first layer, with the anion exchange particles provided as a second layer.

The material described above may also further comprise organic compounds absorber. The organic compounds absorber may be intermixed with one or more of the other materials to form an intermixed layer or it may form a separate layer. The organic compounds absorber may be selected from the group consisting, amongst others, of activated carbons, molecular sieves, zeolites and diatomaceous earth. The organic compounds absorber particles may be activated carbon particles. In one embodiment, the organic compound absorber in the primary layer may be an activated carbon filter pad. In another embodiment, the organic compound absorber comprises activated carbon particles.

The activated carbon particles may have an average particle size in the range of from about microns to about 1000 microns, about 10 microns to about 250 microns, about 20 microns to about 200 microns, about 25 microns to about 150 microns, about 50 microns to about 100 microns, about 25 microns to about 250 microns or from about 100 microns to about 200 microns or from about 100 microns to about 150 microns or from about 150 microns to about 300 microns or from about 200 microns to about 300 microns or from about 400 microns to about 900 microns or from about 500 microns to about 800 microns or from about 600 microns to about 700 microns or from about 250 microns to about 500 microns or from about 250 microns to about 1000 microns.

In one embodiment, the activated carbon particles may be replaced by any particles that are able to absorb organic compounds. Preferably, the particles are able to absorb organic compounds and/or organic metabolites selected from the group comprising creatinine, uric acid and other small and medium sized organic molecules without releasing anything in exchange. The activated carbon particles may also be physically compacted into a predetermined volume for the purpose of space economy. In one embodiment, the activated carbon particles are physically compacted into an activated carbon filter pad.

When the organic compounds absorber is present as part of the material, it may be present in an amount of from 10 to 40 wt % relative to the total weight of the components listed in the broadest version of the material described above (i.e. the material which contains from 30 to 79 wt % of an acidic and/or a neutral zirconium phosphate; from 20 to 65 wt % of an alkaline hydrous zirconium oxide; from 0.1 to 10 wt % of CaCO₃ and/or MgCO₃; and from 0 to 5 wt % of Ca(OH)₂). For example, the organic compounds absorber may be present in an amount of from 15 to 25 wt %, such as from 18 to 23 wt %, such as from 19 to 21 wt % relative to the total weight of the components listed in the broadest version of the material described above.

The materials disclosed herein may also further comprise a neutral hydrous zirconium oxide, which may be obtained by analogy to the process described herein for the production of alkaline hydrous zirconium oxide. When present in the compositions described herein, the neutral hydrous zirconium oxide may be present in an amount of from 0.1 to 10 wt % relative to the total weight of the components in the broadest version of the material described above (i.e. the material which contains from 30 to 79 wt % of an acidic and/or a neutral zirconium phosphate; from 20 to 65 wt % of an alkaline hydrous zirconium oxide; from 0.1 to 10 wt % of CaCO₃ and/or MgCO₃; and from 0 to 5 wt % of Ca(OH)₂). For example, the neutral hydrous zirconium oxide may be present in an amount of from 0.5 to 5 wt % relative to the total weight of the components listed in the broadest version of the material described above.

The neutral hydrous zirconium oxide may be intermixed with one or more of the other materials to form an intermixed layer or it may form a separate layer. For example, it may be mixed with the alkaline hydrous zirconium oxide. Neutral hydrous zirconium oxide can be used as an alternative to alkaline hydrous zirconium oxide, with similar balance outcome. However, neutral hydrous zirconium oxide may add chloride ions to the patient and hence the use of alkaline hydrous zirconium oxide is preferred over neutral hydrous zirconium oxide. Nevertheless, an appropriate amount of neutral hydrous zirconium oxide may be added to the sorbent material.

In certain embodiments of the invention, the CaCO₃ and/or MgCO₃ mentioned in the materials described herein may be only CaCO₃.

In certain embodiments that may be described herein, the acidic and/or water-insoluble metal phosphate may be an acidic zirconium phosphate. In alternative embodiments, the acidic and/or water-insoluble metal phosphate may be an acidic zirconium phosphate and a neutral zirconium phosphate. Any suitable ratio of the acidic and neutral zirconium phosphates may be used herein. Examples of suitable ratios include, but are not limited to situations where the acidic zirconium phosphate is present in an amount of from 55 to 80 wt % of the total amount of zirconium phosphate in the material, with the neutral zirconium phosphate supplying the balance to 100 wt %. For example, the acidic zirconium phosphate may be present in an amount of from 59 to 70 wt % of the total amount of zirconium phosphate in the material, with the neutral zirconium phosphate supplying the balance to 100 wt %; or the acidic zirconium phosphate may be present in an amount of from 75 to 78 wt % of the total amount of zirconium phosphate in the material, with the neutral zirconium phosphate supplying the balance to 100 wt %.

As will be appreciated, the components of the material for use in sorbent-based dialysis presented herein may be provided as individual layers or may be intermixed together in any suitable manner. In particular embodiments of the invention, all of the materials may be intermixed together to provide a single layer of material. In alternative embodiments of the invention, the CaCO₃ and/or MgCO₃, and, when present, Ca(OH)₂ may be intermixed with the acidic and/or neutral zirconium phosphate to form a first layer, with alkaline hydrous zirconium oxide provided as a second layer.

It is noted that CaCO₃ and/or MgCO₃, and, when present, Ca(OH)₂ (and the equivalent materials mentioned herein—i.e.: an alkali metal carbonate, a water insoluble alkaline earth metal carbonate, and a water insoluble polymeric ammonium carbonate and Mg(OH)₂) may cause problems if each is presented as a single homogeneous layer. This is because these materials may form a very dense sludge when presented as a homogenous layer, resulting in restricted flow through a sorbent cartridge. As such, it may be preferred to mix these materials with at least one of alkaline zirconium phosphate and hydrous zirconium oxide (activated carbon or other organic compounds absorber materials may also be intermixed when it is present in the sorbent).

It will be appreciated that the materials disclosed herein may be provided in a sorbent cartridge and may be arrange accordingly within the cartridge to provide the desired effects mentioned herein. That is, the materials that form part of the sorbent may be provided as a single homogeneously mixed layer or as two separate layers, as discussed above.

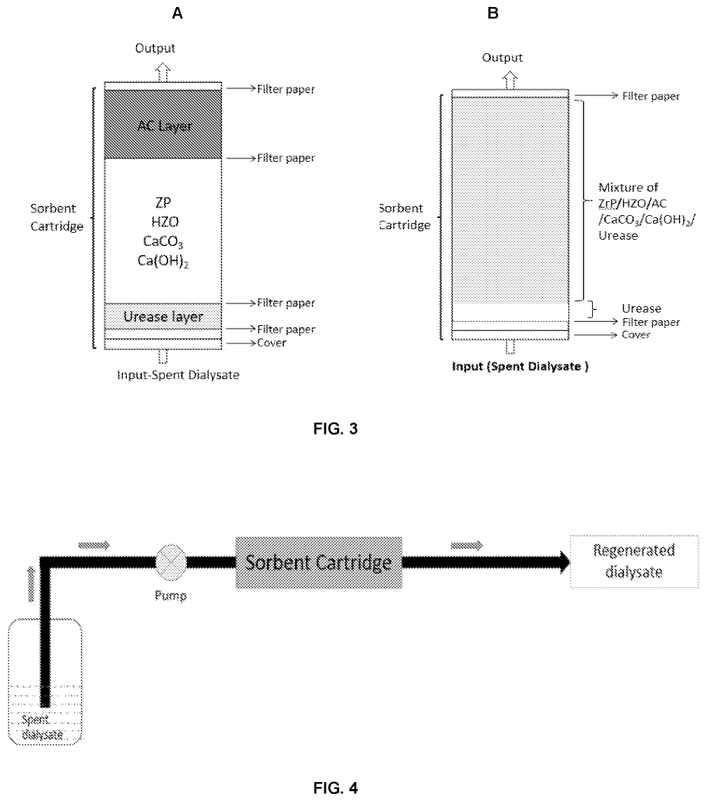

Examples of arrangements that may be used include, but are not limited to those depicted in FIGS. 3 and 6-8.

FIG. 3A depicts an arrangement where the sorbent cartridge 300 contains the materials described herein in a single intermixed layer 310, sandwiched between a urease layer 320 and an activated carbon layer 330. Each layer is separated from the others by a filter paper 340.

FIG. 3b shows a different arrangement where the materials are also intermixed with a portion of the urease present in the sorbent 350 as well as a separate urease layer 340.

It will be noted that the dialysate is intended to enter the cartridge at the end closet to the urease 360 and exit from the end furthest from the urease 370 in both arrangements.

When used herein, the term “urease” is a synonym for the term “uremic toxin-treating enzyme” and both refer to an enzyme able to react with a uremic toxin as a substrate. For example, the uremic toxic-treating enzyme may be an enzyme able to react with urea as a substrate, with uric acid as a substrate, or with creatinine as a substrate. Uremic enzymes can be determined to have this function in vitro, for example, by allowing the enzyme to react with a uremic toxin in solution and measuring a decrease in the concentration of the uremic toxin. Examples of uremic toxin-treating enzymes include, but are not limited to, ureases (which react with urea), uricases (which react with uric acid), or creatininases (which react with creatinine).

The term “uremic toxin” as used herein refers to one or more compounds comprising waste products, for example, from the breakdown of proteins, nucleic acids, or the like, as would be well understood by the person skilled in the art. Non-limiting examples of uremic toxins include urea, uric acid, creatinine, and beta-2 (β₂) microglobulin. In healthy individuals, uremic toxins are usually excreted from the body through the urine. However, in certain individuals, uremic toxins are not removed from the body at a sufficiently fast rate, leading to uremic toxicity, i.e. a disease or condition characterized by elevated levels of at least one uremic toxin with respect to physiologically normal levels of the uremic toxin. Non-limiting examples of disorders associated with uremic toxins include renal disease or dysfunction, gout, and uremic toxicity in subjects receiving chemotherapy.

The term “uremic toxin-treating enzyme particles” as used herein refers to a uremic toxin-treating enzyme in particle form. The enzymes may be immobilized by way of a covalent or physical bond to a biocompatible solid support, or by cross-linking, or encapsulation, or any other means.

The uremic toxin-treating enzyme may be immobilized on any known support material, which can provide immobilization for the uremic toxin-treating enzyme particles. Immobilization may be by physical means such as by adsorption on alumina. In an embodiment non-immobilised enzyme is used. Alternatively, other methods are used to convert urea to ammonia.

In one embodiment, the support material is a biocompatible substrate to which the enzyme is covalently bound. The biocompatible material may be a carbohydrate-based polymer, an organic polymer, a polyamide, a polyester, or an inorganic polymeric material. The biocompatible substrate may be a homogeneous substrate made up of one material or a composite substrate made up of at least two materials. The biocompatible substrate may be at least one of cellulose, Eupergit, silicon dioxide (e.g. silica gel), zirconium phosphate, zirconium oxide, nylon, polycaprolactone and chitosan.

In one embodiment, the immobilization of the uremic toxin-treating enzyme on the biocompatible substrate is carried out by immobilization techniques selected from the group consisting of glutaric aldehyde activation, activation with epoxy groups, epichlorohydrin activation, bromoacetic acid activation, cyanogen bromide activation, thiol activation, and N-hydroxysuccinimide and diimide amide coupling. The immobilization techniques used may also involve the use of silane-based linkers such as (3-aminopropyl) triethoxysilane, (3-glycidyloxypropyl) trimethoxysilane or (3-mercaptopropyl) trimethoxysilane. The surface of the biocompatible substrate may be further functionalized with a reactive and/or stabilizing layer such as dextran or polyethyleneglycol, and with suitable linker- and stabilizer molecules such as ethylenediamine, 1,6-diaminohexane, thioglycerol, mercaptoethanol and trehalose. The uremic toxin-treating enzyme can be used in purified form, or in the form of crude extract such as extract of urease from Jack Bean or other suitable urease sources.

The uremic toxin-treating enzyme particles may be capable of converting urea to ammonium carbonate. In one embodiment the uremic toxin-treating enzyme is at least one of urease, uricase and creatininase. In a preferred embodiment, the uremic toxin-treating enzyme is urease.

In one embodiment, the uremic toxin-treating enzyme particles are urease particles.

In one embodiment the uremic toxin-treating enzyme particles have an average particle size in the range of from about 10 microns to about 1000 microns, about 100 microns to about 900 microns, about 200 microns to about 900 microns, about 300 microns to about 800 microns, about 400 microns to about 700, 500 microns to about 600 microns, about 25 microns to about 250 microns, about 25 microns to about 100 microns, about 250 microns to about 500 microns, about 250 microns to about 1000 microns, about 125 microns to about 200 microns, about 150 microns to about 200 microns, about 100 microns to about 175 microns, and about 100 microns to about 150 microns.

In one embodiment, 1000 to 10000 units of urease are immobilized on said biocompatible substrate. The overall weight of immobilized urease and the substrate ranges from about 0.5 g to about 30 g.

FIG. 6 depicts a further sorbent cartridge 600 according to the invention, where the CaCO₃ and Ca(OH)₂ (when present) are mixed together with hydrous zirconium oxide to form a layer 610 sandwiched between a layer of activated carbon 620 and a layer of zirconium phosphate 630 (as according to the invention). A separate layer of urease 640 is also present and each layer is separated by a filter paper 650. The dialysate is intended to enter via port 660 and exit via port 670 in the cartridge 600.

FIG. 7 depicts a further sorbent cartridge 700 according to the invention, where CaCO₃ is mixed together with zirconium phosphate to form layer 710, Ca(OH)₂ (when present) is intermixed with hydrous zirconium oxide to form a layer 720 sandwiched between a layer of activated carbon 730 and the layer of CaCO₃ and zirconium phosphate 710. A separate layer of urease 740 is also present and each layer is separated by a filter paper 750. The dialysate is intended to enter via port 760 and exit via port 770 in the cartridge 700.

FIG. 8 depicts a further sorbent cartridge 800 according to the invention, where the CaCO₃ and Ca(OH)₂ (when present) are mixed together with zirconium phosphate (as according to the invention) to form layer 810. This layer is sandwiched between a layer of activated carbon 820 and a layer of hydrous zirconium oxide 830. A separate layer of urease 840 is also present and each layer is separated by a filter paper 850. The dialysate is intended to enter via port 860 and exit via port 870 in the cartridge 800.

Further aspects and embodiments of the invention will now be discussed by reference to the following non-limiting examples.

### EXAMPLES

### Materials and Methods

All chemicals (NaCl, NaHCO₃, CaCl₂·2H₂O, MaCl₂·6H₂O, KCl, Glucose monohydrate, Urea, Creatinine and NaH₂PO₄·2H₂O) for synthetic dialysate preparation, and CaCO₃ and Ca(OH)₂ were purchased from Sigma-Aldrich (USA). Amorphous Acidic Zirconium Phosphate (pH: 3.8-4.3), Amorphous Neutral Zirconium Phosphate (pH: 5.8-6.1), Amorphous Hydrous Zirconium Oxide (pH: 11.0-11.5), and immobilised urease were prepared as described below.

Powdered activated carbon (NDS Centaur) was purchased from Calgon Corporation. All the reagents and material were used without further purification. pH of all the samples were recorded with Sartorius PB-10 bench top pH meter. All the analytes (sodium, bicarbonate, urea, ammonia etc.) concentrations were measured with Vitros-250 chemistry analyzer.

### Preoaration 1: Preparation of Zirconium Phosphate

Zirconium phosphate was synthesised by conventional methods, for example by reaction of an aqueous mixture of Basic Zirconium Sulfate and phosphoric acid as described in U.S. Pat. No. 3,850,835. Alternatively, it was synthesised from an aqueous mixture of Sodium Zirconium Carbonate and phosphoric acid as described in U.S. Pat. No. 4,256,718.

The product was titrated to a solution pH of 3.8 to 6.1. A 5M solution of sodium hydroxide was added step-wise to an aqueous slurry of the zirconium phosphate until the desired pH was reached. After the titration, the zirconium phosphate was washed until the filtrate was within acceptable limits of leachables, and air dried.

### Preoaration 2: Preparation of Hydrous Zirconium Oxide

Hydrous zirconium oxide was synthesised by conventional methods, for example by reaction of an aqueous mixture of sodium zirconium carbonate and sodium hydroxide as described in U.S. Pat. No. 4,256,718. This was done by making an aqueous slurry of the hydrous zirconium carbonate and titrating it with 5M sodium hydroxide until the slurry is at a pH of 11 to 12. In some instances, the hydrous zirconium oxide was then washed until the concentration of leachable in the filtrate was within acceptable levels, and air dried.

### Example 1

### Preparation of Sorbent Cartridges

The sorbent cartridge consisted of the materials listed below in Tables 1-3. Zirconium phosphate (ZP) was prepared according to Preparation 1. Hydrous zirconium oxide (HZO) was prepared as described in Preparation 2. Immobilised urease (IU) was prepared as described in Examples 1 and 2 of WO 2011/102807, the contents of which are incorporated herein by reference. Activated carbon (AC) having a particle size of 50 to 200 micron was used. Calcium carbonate (CaCO₃) and calcium hydroxide (Ca(OH)₂) were purchased commercially and had a particle size range of 1 to 100 μm. The sorbent cartridge used to obtain the experimental results below consisted of an empty polypropylene flash column packed with the above sorbent materials (FIG. 3).

TABLE 1

| Composition | A (Comp) | | B | | C | | D | | E | |
|---|---|---|---|---|---|---|---|---|---|---|
| Acidic ZP | 165 | g | 165 | g | 165 | g | 145.2 | g | 145.2 | g |
| Neutral ZP | — | | — | | — | | 36.3 | g | 36.3 | g |
| Alkaline | 165 | g | 165 | g | 165 | g | 148.5 | g | 148.5 | g |
| HZO | | | | | | | | | | |
| Activated | 75 | g | 73 | g | 71.9 | g | 70 | g | 70 | g |

| Carbon | | | | | | | | | |
|---|---|---|---|---|---|---|---|---|---|
| CaCO₃ | — | 2 | g | 3.1 | g | 3 | g | 2 | g |

| | | | | | | | |
|---|---|---|---|---|---|---|---|
| Ca(OH)₂ | — | — | — | 2.5 | g | 2.5 | g |

TABLE 2

| Composition | F | | G | | H | | I | | J | |
|---|---|---|---|---|---|---|---|---|---|---|
| Acidic ZP | 111.1 | g | 111.1 | g | 111.1 | g | 111 | g | 111 | g |
| Neutral ZP | 51.5 | g | 51.5 | g | 51.5 | g | 51.5 | g | 51.5 | g |
| Alkaline | 162.5 | g | 162.5 | g | 162.5 | g | 162.5 | g | 162.5 | g |
| HZO | | | | | | | | | | |
| Activated | 75 | g | 75 | g | 75 | g | 71 | g | 69 | g |
| Carbon | | | | | | | | | | |
| CaCO₃ | 2 | g | 4 | g | 6 | g | 4 | g | 6 | g |

| | | | | | |
|---|---|---|---|---|---|
| Ca(OH)₂ | — | — | — | — | — |

TABLE 3

| | | |
|---|---|---|
| Composition | K | L |

| | | | |
|---|---|---|---|
| Acidic ZP | — | 117 | g |

| | | | | |
|---|---|---|---|---|
| Neutral ZP | 195 | g | 61.75 | g |
| Alkaline | 130 | g | 146.25 | g |

HZO

| | | | | |
|---|---|---|---|---|
| Activated | 73 | g | 69 | g |

Carbon

| | | | | |
|---|---|---|---|---|
| CaCO₃ | 2 | g | 6 | g |

| | | |
|---|---|---|
| Ca(OH)₂ | — | — |

The immobilized urease catalyses the hydrolysis of urea into ammonia and carbon dioxide. Zirconium phosphate acts as cation exchanger and releases back Na⁺ or H⁺ in exchange of Ca⁺⁺, Mg⁺⁺ and NH₄⁺. Hydrous zirconium oxide acts as an amphoteric ion exchanger that mainly binds negatively charged species like phosphate and fluoride. Additives CaCO₃ and Ca(OH)₂ function as a source of carbonate and alkali and helps to maintain the pH and bicarbonate balance in desired range. The activated charcoal, a highly micropormus material with an exceptionally high surface area, adsorbs heavy metals, small water-soluble uremic toxins like creatinine and uric acid, middle molecules such as B2-microglobulin, and protein-bound uremic toxins. The sorbent cartridges and sorbent materials were prepared as described below.

In the examples used herein, the column was packed with:
1) AC layer, followed by a filter paper separator;
2) a mixture of ZP, HZO and CaCO₃/Ca(OH)₂, followed by a filter paper separator; and
3) a layer of immobilised urease.

The column was then inverted and installed in the experimental setup in such a way that spent dialysate flowed into the IU layer first and exited via the AC layer.

As will be appreciated, the cartridge may make use of different configurations of intermixing and ordering among the layer(s) (FIGS. 3 and 7 to 9).

### General Procedure 1

Compositions A to H were tested using a proprietary method referred to hereinafter as “General Procedure 1”. This proprietary method involved the pumping of two different solutions through a sorbent at dynamic mixing ratios calculated to more accurately mimic the changing composition of dialysate during normal use in vivo. Between them, the solutions comprise a mixture of sugars, salts, toxins (e.g. urea, creatinine, phosphate and other toxins) blended at proprietary ratios. The use of a dynamic dialysate solution is believed to provide more accurate results than traditional simulated dialysate solutions, enabling more accurate testing of sorbents.

The balances for the key electrolyte like sodium and bicarbonate was obtained according to below formula

$$Sodium balance = ( {C}_{Na Drain} - {C}_{Na pre} ) * {V}_{drain}$$

$$Bicarbonate balance = {C}_{HCO 3 Drain} * {V}_{drain} - {C}_{HCO 3 SD} * {V}_{SD used}$$

### Where

C_{Na Drain}=Concentration of sodium in collected fluid at the end of experimentC_{Na pre}=Average Concentration of sodium in synthetic dialysate
V_{drain}=Volume of the fluid collected at end of experiment
C_{HCO3 Drain}=Concentration of bicarbonate in collected fluid at the end of experiment
C_{HCO3 SD}=Concentration of bicarbonate in synthetic dialysate
V_{SD used}=Volume of the synthetic dialysate containing bicarbonate used for experiment

### Example 2

Compositions A, B and C from Example 1 were used in General Procedure 1 using a urea input of from 7.9 to 8.6 mM to produce the results in Table 4.

TABLE 4

| | | HCO3 | Urea | | |
|---|---|---|---|---|---|
| | Na bal | Bal | Input | Urea | Coll |
| Composition | (mmol) | (mmol) | mM) | cleared(g) | vol(L) |
| (A) Acidic ZP (165 g)/ | −16.3 | −18.9 | 8.6 | 8 | 14.1 |
| Alkaline HZO | | | | | |
| (165 g)/AC (75 g) | | | | | |
| (B) Acidic ZP (165 g)/ | −10.1 | −4.9 | 8.4 | 7 | 14.0 |
| Alkaline HZO | | | | | |
| (165 g)/AC (73 g) + | | | | | |
| 2 gCaCO₃ | | | | | |
| (C) Acidic ZP(165 g)/ | 8.1 | −0.3 | 7.9 | 6.6 | 14.0 |
| Alkaline HZO | | | | | |
| (165 g)/AC (71.9 g) + | | | | | |
| CaCO₃ (3.1 g) | | | | | |

Negative balance indicates removal from dialysate

Compositions B and C are “high urea” cartridges and were prepared by mixing acidic zirconium phosphate with hydrous zirconium oxide in equal proportion with varying amounts of calcium carbonate (Composition A is a comparative example, with no CaCO₃). The desired sodium and bicarbonate balance can be achieved by adjusting the amount of calcium carbonate, as can be seen in Table 4, where a better bicarbonate balance was obtained by increasing the amount of calcium carbonate from 0 g to 3.1 g.

As can be observed from Table 4's data, the sequential increase in CaCO₃ content is accompanied by an increase in bicarbonate balance, as it acts as source of HCO₃₋ ions and sodium balance, because calcium is preferably bound by the ZP, leaving less capacity for other cations.

### Example 3

Compositions D and E from Example 1 were used in General Procedure 1 using a urea input of 8.1 mM to produce the results in Table 5.

TABLE 5

| | Ammonia |
|---|---|
| Composition | removed (mmol) |
| (D) Acidic ZP (145.2 g)/Neutral ZP | 245 |
| (36.3 g)/Alkaline HZO (148.5 g)/ | |
| AC (70 g) + CaCO3 (3 g) + Ca(OH)2 (2.5 g) | |
| (E) Acidic ZP (145.2 g)/Neutral ZP | 255 |
| (36.3 g)/Alkaline HZO (148.5 g)/ | |
| AC (70 g) + CaCO3 (2 g) + Ca(OH)2 (2.5 g) | |

The amount of ammonia removed was calculated by multiplying the amount of urea removed by 17 and the amount of urea removed was calculating by multiplying the difference of input and output urea concentration (mmol/I) and amount of fluid that had passed through the cartridge (14 L). Above data shows that increase in CaCO3 amount added to the sorbent by 1 g (approx. 10 mmol) reduces ammonia binding by 10 mmols.

### Example 4

Compositions F, G and H from Example 1 were used in General Procedure 1 using a urea input of from 5.0 to 5.2 mmol/L to produce the results in Table 6.

TABLE 6

| | | | | | Coll |
|---|---|---|---|---|---|
| | Na | HCO₃ | Urea | Urea | vol |
| Sorbent Composition | bal | bal | Input | cleared(g) | (L) |
| (F) Acidic ZP (111.1 g)/ | −18 | −21 | 5.1 | 4.3 | 14.0 |
| Neutral ZP (51.5 g)/ | | | | | |
| Alkaline HZO (162.5 g)/ | | | | | |
| AC (75 g) + CaCO3 | | | | | |
| (2 g) | | | | | |
| (G) Acidic ZP (111.1 g)/ | −10 | −8 | 5.2 | 4.4 | 14.0 |
| Neutral ZP (51.5 g)/ | | | | | |
| Alkaline HZO (162.5 g)/ | | | | | |
| AC (75 g) + CaCO3 | | | | | |
| (4 g) | | | | | |
| (H) Acidic ZP (111.1 g)/ | −15 | 22 | 5.0 | 4.3 | 14.1 |
| Neutral ZP (51.5 g)/ | | | | | |
| Alkaline HZO (162.5 g)/ | | | | | |
| AC (75 g) + CaCO3 | | | | | |
| (6 g) | | | | | |

Compositions F-H may be considered to form “Low urea” cartridges intended to deal with a urea load of from 3-5 mmol/L.
The sequential increase in CaCO₃ content is accompanied by an increase in bicarbonate balance.
However, sodium balance was less affected in this case in comparison with compositions used in Example 2 due to the lower amount of AZP. AZP can adsorb more sodium and ammonium ions because it contains H⁺ ions, so a reduction in AZP may explain this difference. Nevertheless, in these compositions, less ammonium ions are released, so a reduced amount of AZP (compared to those used in Examples 2 and 3) is sufficient to maintain the desired sodium and bicarbonate balances.

### Example 5

Compositions I and J from Example 1 were used in General Procedure 1 using a urea input of from 2.3 to 5.2 mM to produce the results in Tables 7 and 8.

TABLE 7

| | | | Urea | | |
|---|---|---|---|---|---|
| | Na bal | HCO₃ bal | Input | Urea | Coll |
| | (mmol) | (mmol) | (mM) | cleared(g) | vol(L) |
| (I) Neutral ZP (51.5 g)/Acidic | −22 | −42 | 3.3 | 2.7 | 14.0 |
| ZP(111 g)/Alkaline HZO | | | | | |
| (162.5)/AC (71 g) + CaCO3 (4 g) | | | | | |
| (I) Neutral ZP (51.5 g)/Acidic | −18 | −27 | 4.3 | 3.6 | 14.0 |
| ZP(111 g)/Alkaline HZO | | | | | |
| (162.5)/AC (71 g) CaCO3 (4 g) | | | | | |
| (I) Neutral ZP (51.5 g)/Acidic | −10 | −8 | 5.2 | 4.4 | 14.0 |
| ZP(111 g)/Alkaline HZO | | | | | |
| (162.5)/AC (71 g) + CaCO3 (4 g) | | | | | |

TABLE 8

| | | HCO3 | Urea | | |
|---|---|---|---|---|---|
| | Na bal | bal | Input | Urea | Coll |
| | (mmol) | (mmol) | (mM) | cleared(g) | vol(L) |
| (J) Neutral ZP (51.5 g)/Acidic ZP | −37 | −15 | 2.3 | 1.9 | 14.0 |
| (111 g)/Alkaline HZO (162.5)/AC | | | | | |
| (69 g) + CaCO3 (6 g) | | | | | |
| (J) Neutral ZP (51.5 g)/Acidic | −33 | −2 | 3.2 | 2.7 | 14.0 |
| ZP(111 g)/Alkaline HZO (162.5)/AC | | | | | |
| (69 g) + CaCO3 (6 g) | | | | | |
| (J) Neutral ZP (51.5 g)/Acidic | −18 | 6 | 4.3 | 3.6 | 14.0 |
| ZP(111 g)/Alkaline HZO (162.5)/AC | | | | | |
| (69 g)] + CaCO3 (6 g) | | | | | |
| (J) Neutral ZP (51.5 g)/Acidic | −15 | 22 | 5.0 | 4.3 | 14.1 |
| ZP(111 g)/Alkaline HZO (162.5)/AC | | | | | |
| (69 g) + CaCO3 (6 g) | | | | | |

As can be seen, the sodium balance and bicarbonate balance increased with urea concentration for these “low urea” cartridges.

### Example 6

As noted above, Examples 2 to 5 were run under proprietary conditions. The input dialysate composition was varied to mimic the dialysate chemistry in the peritoneal environment. To this end, the initial dialysis fluid was substantially a fresh dialysate of pH 5.2, whereafter the input dialysate was progressively altered to a synthetic spent dialysate with pH 7.4.

During experiments a maximum pH of 7.5 was attained. Having incorporated a mechanism to improve on pH, its level is not without limit. While designing a new sorbent configuration, its output pH has to fall in between 5-8 in order to be physiologically acceptable. In addition to considerations regarding metabolic acidosis, low pH level can result in high pCO₂ (partial CO₂ pressure) level in the dialysate, which occurs concurrently with dissolved CO₂. Exposure of the patient to high pCO₂ dialysate could result in gas formation in the peritoneum (pneumoperitoneum), a possible cause of abdominal pain and discomfort.

The composition of CaCO₃ and Ca(OH)₂ described herein will directly affect the final balance performance of the sorbent in terms of Na⁺ and HCO₃₋ balance as well as the pH which impacts pCO₂ level.

The following further compositions were prepared and loaded onto cartridges according to General Procedure 1 (proprietary method).
1. Acidic ZP (145.2 g)/Neutral ZP (36.3 g)/Alkaline HZO (148.5)/AC (70 g) Ca(OH)₂ (4 g)
2. Acidic ZP (145.2 g)/Neutral ZP (36.3 g)/Alkaline HZO (148.5)/AC (70 g) 4 g CaCO₃1 g Ca(OH)₂
3. Acidic ZP (145.2 g)/Neutral ZP (36.3 g)/Alkaline HZO (148.5)/AC (70 g) 3 g CaCO3 1.75 g Ca(OH)2

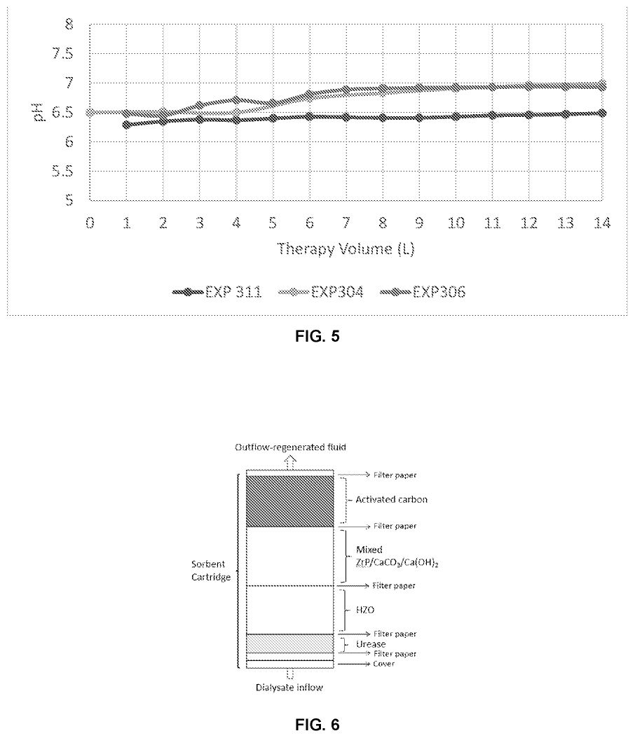

FIG. 5 demonstrates the effect of different quantities of Ca(OH)₂ on the pH profile during a simulated 14 L therapy run. As the amount of Ca(OH)₂ is increased (Exp 311 vs Exp 304/306), the effect of increase in pH is more prolonged. Since it takes time for urea to diffuse from blood to dialysate, it would be expected that CO₂ generated from urea hydrolysis will increase during the latter part of the dialysis treatment. Therefore, it is desirable that the pH profile is increased in the second half of the treatment as well, in order to maximize the effect of Ca(OH)₂ on HCO-balance.

### General Procedure 2

Further experiments were performed using the following procedure, in which synthetic spent dialysate of known electrolyte and toxin concentration is pumped through a cartridge containing sorbent material at a constant flow rate (FIG. 4).

### Preparation of the Synthetic Dialysate:

14 L of synthetic dialysate was used for single pass experiments, using the setup shown in FIG. 4 with concentrations of the various cations and anions in the synthetic dialysate provided in Table 9. Urea was added according to the desired final urea concentration and it typically ranges in concentration between 3 mmol/L-8 mmol/L

TABLE 9

| Target Values | Conc (mmol/L) |
|---|---|
| Sodium | 132 |
| Bicarbonate | 20 |
| Lactate | 15 |
| Calcium | 1.25 |
| Magnesium | 0.25 |
| Potassium | 2.7 |
| Glucose (%) | 1.5 |
| CI (resulting) | 101.85 |

The synthetic dialysate having the above concentrations was prepared by mixing salts in the amounts described in Table 10 below. The pH of the synthetic dialysate was adjusted to 7.4-7.6 by adding 5N HCl.

TABLE 10

| | Molar | | Amount |
|---|---|---|---|
| | Mass | Concentration | required |
| | (g/mol) | (mmol/L) | (g) |
| Sodium Chloride | 58.44 | 96.15 | 78.67 |
| Sodium Bicarbonate | 84.00 | 20.00 | 23.52 |
| Sodium L-Lactate | 112.06 | 60% | 39.22 |
| solution (g) | | aqueous | |
| | | solution | |
| Calcium Chloride dihydrate | 147.01 | 1.25 | 2.57 |
| Magnesium chloride | 203.30 | 0.25 | 0.71 |
| hexahydrate | | | |
| Potassium Chloride | 74.55 | 2.70 | 2.82 |
| Glucose | 180.16 | 75.70 | 190.93 |
| Urea | 60.06 | 5.50 | 4.62 |
| Creatinine | 113.12 | 0.50 | 0.79 |
| NaH2PO4*2H2O | 156.01 | 0.85 | 1.86 |

### Example 7

Four experiments were conducted under single pass conditions (General Procedure 2) using low urea cartridges to demonstrate the effect of input urea concentration and significance of calcium carbonate in managing bicarbonate balance and sodium balance. The composition of the sorbents tested is shown in Table 11.

TABLE 11

| Composition | Experiment 1 | | Experiment 2 | | Experiment 3 | | Experiment 4 | |
|---|---|---|---|---|---|---|---|---|
| Acidic ZP | 129 | g | 129 | g | 129 | g | 129 | g |
| Neutral ZP | 57.8 | g | 57.8 | g | 57.8 | g | 57.8 | g |
| Alkaline HZO | 153 | g | 153 | g | 153 | g | 153 | g |
| Activated Carbon | 75 | g | 75 | g | 75 | g | 75 | g |
| CaCO₃ | 0 | g | 2 | g | 6.5 | g | 6.5 | g |
| Ca(OH)₂ | 0 | g | 0 | g | 0 | g | 0 | g |
| Urea | 5.43 | mM | 5.43 | mM | 5.61 | mM | 3.0 | mM |

| | | | | |
|---|---|---|---|---|
| Sodium Balance | −42.5 | −22 | 9 | 4.5 |
| Bicarbonate | −83 | −45 | −10 | −32 |
| Balance | | | | |

Experiment 1 was carried out using a base formulation for Low Urea Cartridge (LUC) without calcium carbonate, and a high negative bicarbonate balance (−83 mmol) was observed because there was no additional source of bicarbonate in form of calcium carbonate.

From Experiment 1 to Experiment 3, the amount of calcium carbonate in the sorbent was increased from 0 g (in Experiment 1) to 6 g (in Experiment 3) while maintaining the base composition of sorbent and input dialysate composition. An increase in average bicarbonate balance is observed from −83 mmol to −10 mmol (column 3), indicating the importance of calcium carbonate in maintaining neutral bicarbonate balance. Increasing the amount of calcium carbonate also led to a higher sodium balance due to release of additional sodium ions exchanged to calcium ions contributed by calcium carbonate.

Experiment 4 was carried out to demonstrate the impact of input urea concentration on bicarbonate balance and sodium balance. Experiments 3 and 4 were conducted under similar conditions with the same sorbent composition. However, the input urea concentration was reduced in Experiment 4 as compared to Experiment 3 (5.61 mmol/L vs 3 mmol/L). A higher sodium balance is observed at higher input urea concentration due to the availability of more exchangeable ammonium ions (from urea) with sodium. Higher urea also contributes to higher bicarbonate balance.

Four further experiments (Experiment 5 to Experiment 8) were carried out using a high urea cartridges and results are provided in Table 12.

TABLE 12

| Composition | Experiment 5 | | Experiment 6 | | Experiment 7 | | Experiment 8 | |
|---|---|---|---|---|---|---|---|---|
| Acidic ZP | 146 | g | 146 | g | 146 | g | 146 | g |
| Neutral ZP | 41 | g | 41 | g | 41 | g | 41 | g |
| Alkaline HZO | 153 | g | 153 | g | 153 | g | 153 | g |
| Activated Carbon | 70 | g | 70 | g | 70 | g | 70 | g |
| CaCO₃ | 0 | g | 2.0 | g | 2.0 | g | 2.0 | g |
| Ca(OH)₂ | 0 | g | 0 | g | 2.5 | g | 2.5 | g |
| Urea | 6.34 | mM | 6.34 | mM | 6.45 | mM | 7.54 | mM |

| | | | | |
|---|---|---|---|---|
| Sodium Balance | 5.6 | 13.4 | 20.1 | 21.5 |
| Bicarbonate | −48.9 | −29.1 | −20.2 | −7.6 |
| Balance | | | | |

It is observed that the trends shown by these results (Experiments 1 to 8 in Example 7) are consistent with those in Examples 2 to 5 using a (more accurate) proprietary method.

The invention claimed is:

1. A material for use in sorbent-based dialysis, the material comprising:
one or both of acidic and neutral cation exchange particles;
alkaline anion exchange particles; and
one or more of an alkali metal carbonate, a water insoluble alkaline earth metal carbonate, and a water insoluble polymeric ammonium carbonate.

2. The material according to claim 1, wherein the material further comprises one or both of Ca(OH)₂, and Mg(OH)₂.

3. The material according to claim 1, wherein the one or both of acidic and neutral cation exchange particles are one or both of an acidic and neutral water-insoluble metal phosphate.

4. The material according to claim 3, wherein the metal is zirconium.

5. The material according to claim 1, wherein the alkaline anion exchange particles comprise an amorphous and partly hydrated, water-insoluble metal oxide in one or more of its: hydroxide-; and carbonate-; and acetate-; and lactate- counter-ion form, wherein the metal is selected from one or more of the group consisting of titanium, zirconium, and hafnium.

6. The material according to claim 1, wherein one or more of the following apply:
(a) the water insoluble alkaline earth metal carbonate is selected from one or more of the group consisting of CaCO₃ and MgCO₃;
(b) the alkali metal carbonate is K₂CO₃; and
(c) the water insoluble polymeric ammonium carbonate is selected from one or more of the group consisting of sevelamer carbonate, polymer-bound tetra-alkyl ammonium carbonate, and 3-(trialkyl ammonium) alkyl functionalised silica gel carbonate.

7. The material according to claim 1, wherein the material comprises:
from 30 to 79 wt % of one or both of acidic and neutral cation exchange particles;
from 20 to 65 wt/o of alkaline anion exchange particles;
one or more of an alkali metal carbonate, a water insoluble alkaline earth metal carbonate, and a water insoluble polymeric ammonium carbonate in a total amount from 0.1 to 10 wt %; and
one or both of Ca(OH)₂, and Mg(OH)₂ in a total amount of from 0 to 5 wt %.

8. The material according to claim 7, wherein the material comprises:
from 31 to 75 wt % of one or both of acidic and neutral cation exchange particles;
from 23 to 63 wt % of alkaline anion exchange particles;
one or more of an alkali metal carbonate, a water insoluble alkaline earth metal carbonate, and a water insoluble polymeric ammonium carbonate in a total amount of from 0.1 to 5 wt %; and
one or both of Ca(OH)₂, and Mg(OH)₂ in a total amount of from 0 to 4 wt %.

9. The material according to claim 7, wherein the material comprises:
from 50 to 64 wt % of one or both of acidic and neutral cation exchange particles;
from 35 to 45 wt % of alkaline anion exchange particles; and
one or more of an alkali metal carbonate, a water insoluble alkaline earth metal carbonate, and a water insoluble polymeric ammonium carbonate a total amount of from 0.3 to 5 wt %.

10. The material according to claim 9, wherein the material comprises:
from 53 to 60 wt % of one ore both of acidic and neutral cation exchange particles;
from 39 to 44 wt % of alkaline anion exchange particles; and
one or more of an alkali metal carbonate, a water insoluble alkaline earth metal carbonate, and a water insoluble polymeric ammonium carbonate a total amount of from 0.5 to 3 wt %.

11. The material according to claim 7, wherein the material comprises:
from 45 to 59 wt % of one or both of acidic and/or neutral cation exchange particles;
from 40 to 54 wt % of alkaline anion exchange particles; and
one or more of an alkali metal carbonate, a water insoluble alkaline earth metal carbonate, and a water insoluble polymeric ammonium carbonate a total amount of from 0.5 to 5 wt %.

12. The material according to claim 11, wherein the material comprises:
from 48 to 56 wt % of one or both of acidic and neutral cation exchange particles;
from 42 to 50 wt % of alkaline anion exchange particles, and
one or more of an alkali metal carbonate, a water insoluble alkaline earth metal carbonate, and a water insoluble polymeric ammonium carbonate a total amount of from 1 to 2 wt %.

13. The material according to claim 7, wherein the material comprises:
from 50 to 70 wt % of one or both of acidic and neutral cation exchange particles;
from 30 to 49 wt % of alkaline anion exchange particles;
from 0.2 to 3 wt % one or more of an alkali metal carbonate, a water insoluble alkaline earth metal carbonate, and a water insoluble polymeric ammonium carbonate; and
one or both of Ca(OH)₂, and Mg(OH)₂ in a total amount of from 0.2 to 2 wt %.

14. The material according to claim 13, wherein the material comprises:
from 53 to 67 wt % of one or both of acidic and neutral cation exchange particles;
from 33 to 46 wt % of alkaline anion exchange particles:
one or more of an alkali metal carbonate, a water insoluble alkaline earth metal carbonate, and a water insoluble polymeric ammonium carbonate a total amount of from 0.2 to 2 wt %; and
one or both of Ca(OH)₂ and Mg(OH)₂ in a total amount of from 0.2 to 1.5 wt %.

15. The material according to claim 1, wherein the material is one in which:
the cation exchange particles are one or both of acidic and a neutral water-insoluble metal phosphate;
anion exchange particles are an alkaline hydrous zirconium oxide; and
the one or more of an alkali metal carbonate, a water insoluble alkaline earth metal carbonate, and a water insoluble polymeric ammonium carbonate is one or both of CaCO₃ and MgCO₃.

16. The material according to claim 1, wherein the material further comprises an organic compounds absorber, wherein the organic compounds absorber is present in an amount of from 10 to 40 wt % relative to the total weight of the components listed in claim 1.

17. The material according to claim 16, wherein the organic compounds absorber is activated carbon.

18. The material according to claim 1, wherein one or both of the following apply:
(a) the material further comprises neutral hydrous zirconium oxide, wherein the neutral hydrous zirconium oxide is present in an amount of from 0.1 to 10 wt % relative to the total weight of the components listed in claim 1; and
(b) (i) all of the components are intermixed together to provide a single layer of material; or
(ii) the one or more of an alkali metal carbonate, a water insoluble alkaline earth metal carbonate, and a water insoluble polymeric ammonium carbonate, and, when present, one or both of Ca(OH)₂ and Mg(OH)₂ are intermixed with the cation exchange particles to form a first layer, with the anion exchange particles provided as a second layer.

19. The material according to claim 4, wherein both an acidic zirconium phosphate and a neutral zirconium phosphate are present and the acidic zirconium phosphate is present in an amount of from 55 to 80 wt % of the total amount of zirconium phosphate in the material, with the neutral zirconium phosphate supplying the balance to 100 wt %.

20. (canceled)

21. A cartridge for use in sorbent dialysis, the cartridge comprising a material as described in claim 1.
